(19) United States
(12) Patent Application Publication
(10) Pub. No.: US 2022/0285036 A1
(43) Pub. Date: Sep. 8, 2022

(54) SYSTEMS AND METHODS FOR INTELLIGENT AND PRIVACY-CENTRIC CONTACT TRACING

(71) Applicant: TITAN HEALTH & SECURITY TECHNOLOGIES, INC., Newport Beach, CA (US)

(72) Inventors: Vic A. Merjanian, Newport Beach, CA (US); Ryan Khalili, Newport Beach, CA (US); Eduardo Juarez, Newport Beach, CA (US); Ed Merjanian, Newport Beach, CA (US); Serene Nasser, Newport Beach, CA (US); Daniel Wallengren, Newport Beach, CA (US)

(21) Appl. No.: 17/508,792

(22) Filed: Oct. 22, 2021

Related U.S. Application Data
(60) Provisional application No. 63/105,134, filed on Oct. 23, 2020.

(51) Int. Cl. G16H 50/80; H04W 4/029; H04W 4/80; G16H 15/00

(52) U.S. Cl. CPC G16H 50/80; H04W 4/029; H04W 4/80; G16H 15/00

(57) ABSTRACT

Systems and methods are provided for organizations to implement a proactive and comprehensive approach to the spread of infectious diseases. Specifically, epidemic response systems and methods configured to acquire anonymized, pseudonymized, data minimized, or encrypted data from mobile and stationary nodes, analyze the acquired data to derive patterns and educated inferences, and engage a display component of mobile nodes to present notifications pertaining to the derived patterns and educated inferences. The acquired data may be used by the disclosed systems and methods to determine (1) a proximity to other nodes and users associated with respective nodes including historical paths/locations exposed to infectious agents, (2) a proximity within or to an area formed via a preexisting and/or ad hoc network of beacons, (3) if a number of nodes surpasses a predetermined threshold within an area, and (4) information related to assets or equipment within an area exposed to infectious agents.

### CROSS-REFERENCE TO RELATED APPLICATIONS

The present application claims priority to U.S. Provisional Patent Application No. 63/105,134, filed Oct. 23, 2020, the content of which is incorporated herein in its entirety.

### TECHNICAL FIELD

The disclosed technology relates generally to obtaining, tracking, and communicating information, and some example embodiments disclosed herein relate to obtaining, tracking, and communicating information regarding infectious agents, and to technology enabling contact tracing, population density monitoring, and asset tracking in connection with an emergency situation or epidemic using various forms of communication.

### BACKGROUND

Contact-tracing applications (“apps”) are designed to let people know if they have been in close contact with someone who has tested positive or later tests positive for an infectious agent (e.g., COVID-19). Various forms and designs of these apps are emerging due to the recent spread of the coronavirus (“COVID-19”) and the failure to effectively address its unprecedented outbreak. An app that enables acceptable contact tracing is particularly relevant to organizations, in part, because the Center for Disease Control (“CDC”) encourages and in some instances may require organizations to notify members that may have been in close contact with another member that has tested positive for COVID-19. In the context of COVID-19, close contact is usually defined as any individual who was within 6 feet of an infected person for at least 15 minutes starting from 2 days before illness onset (or prior to positive pathogen collection for asymptomatic individuals) until the time the individual is isolated or recovered. Notably, the thresholds that define close contact can vary and correspond to the characteristics of an infectious disease or pathogen of interest.

Conventional contact-tracing apps are mobile-to-mobile solutions that are highly dependent upon GPS accuracy track interactions and physical proximity (i.e., close contact) between individuals using their mobile devices.

Key problems exist, however, with the mobile-to-mobile solutions. In just one example, among others, the usefulness and granularity of the data is limited by the accuracy of the GPS location technology used in both mobile devices in any given scenario. For example, GPS technology in a typical smartphone is only accurate to within 3-5 meters (e.g. to within 9-15 feet). Thus, for example, where the GPS technology in two mobile phones is relied upon to determine if a “contact” has occurred between the holders of those mobile phones, there will be many false positive and false negative “contacts” whenever the relevant distance threshold for contact tracing purposes is less than twice the accuracy distance (accounting for both mobile phones) achieved by the GPS technology. That is, whenever the relevant distance threshold is less than 18-30 feet (i.e., 9-15 feet×2 mobile phones=18-30 feet), the accuracy of the mobile-to-mobile solutions do not provide sufficient certainty to the contact-tracing objectives. Moreover, because in some solutions an objective of the contact-tracing effort is to continuously track contacts from one person to the next to flag exposures as people come in contact with one another throughout the day, week, etc., even just one false positive and/or one false negative may be propagated throughout the results in a way that significantly undermines the accuracy of data collected. In some cases, false data propagated across a sizeable population can exacerbate the negative effects of an emergency to a far greater degree than they might otherwise play out, thereby causing rather than reducing problems. A false positive diagnosis can both decrease the accuracy of the associated contact tracing app as well as impact the individual and the contacts of the individual falsely identified as infected. Naturally, adversely impacted individuals will be less likely to adopt apps that utilize contact tracing. On the other hand, a false negative diagnosis can present a greater threat to the welfare of the general public. That is, unknowing individuals that are infected may fail to satisfy necessary quarantine protocols associated with a positive diagnosis.

Accordingly, solutions are needed to enhance the accuracy and reliability of contact tracing technologies (e.g., contact tracing apps) to reduce, predict, and model the progression of an infectious disease or pathogen of interest. More accurate and reliable contact tracing, enabled by such solutions, will also increase the likelihood of adoption by the general public, boost the transfer of reliable disease knowledge in a community, and promote adherence to quarantine and/or sanitization procedures related to the infectious disease or pathogen of interest.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

Some of the figures included herein illustrate various embodiments of the disclosed technology from different viewing angles. Although the accompanying descriptive text may refer to such views as “top,” “front,” “back,” “bottom” or “side” views, such references are merely descriptive and do not imply or require that the disclosed technology be implemented or used in a particular spatial orientation unless explicitly stated otherwise.

FIG. 1 is a block diagram illustrating an example exposure response management system in accordance with one or more embodiments of the present disclosure.

FIG. 2 illustrates an example environment in which one or more embodiments of the technology disclosed herein may be implemented.

FIG. 3 is a block diagram illustrating example resources of the exposure response management system in accordance with one or more embodiments of the present disclosure.

FIG. 4 is a flow diagram illustrating an example method in accordance with one or more embodiments of the present

FIG. 5 is a flow diagram illustrating an example method in accordance with one or more embodiments of the present disclosure.

FIG. 6 is a flow diagram illustrating an example method in accordance with one or more embodiments of the present disclosure.

FIG. 7 is a flow diagram illustrating an example system in accordance with one or more embodiments of the present disclosure.

FIG. 8 illustrates an example computing component that may be used in implementing various features of embodiments of the disclosed technology.

The figures are not intended to be exhaustive or to limit the disclosed technology to the precise form disclosed, and that the disclosed technology be limited only by the claims and the equivalents thereof.

### DETAILED DESCRIPTION

In health emergency situations it is often difficult to maintain communication. It can be helpful to provide prompt updates (e.g., real time or near real-time) as a situation develops or as operations return to normal. Relaying health information can prevent an outbreak and help minimize the impact a contagious disease related epidemic or pandemic, such as the coronavirus (COVID-19), can have on a population and/or operations of organizations. Preventative and remedial measures may be communicated to address emergent or ongoing infectious diseases. As the infectious agent spreads, new areas may become exposed and thus riskier for people to travel to these areas. Mass communication can help keep individuals up to date on potential risks and prevent the further spread of disease. However, in general, there is a lack of reliable information available to such individuals when they may be in proximity to infectious agents. In such situations, it can also be difficult for organizations to coordinate efforts to dispatch individual updates, or group updates, in an efficient and effective manner to assist the organization's members that may be infected and/or may have been exposed to an infectious agent. The present disclosure addresses the deficiencies of conventional systems, and provides multi-layer contact tracing and population density monitoring that aids organizations in: (1) identifying locations of infected individuals and exposed individuals (including real-time or near real-time location data); (2) identifying path information showing such individual's trail to arriving at their present location; (3) identifying locations of persons, equipment, assets, or other resources that may be exposed or have been exposed to an infectious agent; (4) establishing communications channels between administrators of organizations and infected persons and/or exposed persons; (5) automatically notifying administrators and individuals when pre-set population density thresholds are exceeded; and (6) various other features that enable more timely and effective tracking of infectious diseases during a health crisis.

Embodiments of the present disclosure provide systems and methods that offer exposure communication solutions to its users which may include public authorities, schools/universities, private companies, non-profits, and various other forms of organizations. Information regarding the status of an exposure-related situation (e.g., epidemic, pandemic) that is tailored to the needs of these organizations may be shared with large groups of people effectively and efficiently. For instance, preventative measures such as tips to avoid infection or emerging data on areas that have confirmed cases of the disease can be relayed via dynamic notifications directed to individuals, groups, and/or an entire community. Further applications of the presently disclosed technology can also be to support an organization's onsite security and work-place safety planning by enrolling their staff, students, personnel, and other individuals associated with these organizations.

Alternative embodiments of the present disclosure may be designed to complement the existing network solutions by utilizing mesh networking over a combination of Wi-Fi® and BLE protocols. That is, mesh topology may be utilized in situations where there are one or more points of failure in an existing network, or even in situations where there is not a single point of failure at all. For example, if all or part of a network goes down due to physical destruction or unexpected congestion of relevant node or nodes, alternative paths or protocols may be activated to relay information. Namely, each device relays information to the next as they communicate with each other. Advantageously, a mesh network allows building a system that is easily adaptable and relocatable and improves the reliability of the network. The integration of mobile devices allows for the creation of networks with variable topology. Moreover, mesh networking provides for interoperability between devices and across platforms. Further details of mesh networking are described in U.S. patent application Ser. No. 16/559,294, filed on Sep. 3, 2019, entitled “GEOFENCED MESH NETWORK COMMUNICATION,” the content of which is incorporated herein by reference.

Some embodiments of the present disclosure may enable an administrator to effectively address an outstanding or emerging crisis (e.g., a health crisis), for example, as applied to a pandemic that is spread by infectious agents such as COVID-19. The disclosed systems and methods may track contacts among and/or between individuals within a designated area and/or designated time frame (e.g., work hours). In one embodiment, the designated area can be formed by a geofence that acts as a virtual boundary for a real-world geographic area. A geofence may be dynamically generated, as in a radius around a point location (e.g., a beacon, a gateway, or other node), and/or a geofence can be a predefined set of boundaries (e.g., several beacons, or a boundary relative thereto). Both methods may be applied to presently disclosed technology at preexisting beacons and/or newly installed beacons at known or recordable locations of interest. In some embodiments, the presently disclosed technology may be used to characterize levels of risk related to particular areas, particular individuals, and/or particular times. Notifications can be transmitted to employees and/or members that are present at locations associated with a sufficiently high level of risk in which alternative routes or alternative meeting places can be suggested. Certain individuals of an organization (i.e., administrators) may be tasked with implementing the notifications based on derived inferences or trends found based on compiled user location and health data.

Related embodiments of the present disclosure describe system that can collect user and health crisis information. This information may include personal identifiers, professional information, employment information, educational information, geolocation information, travel information, race information, gender information, regular route information, underlying health conditions, known susceptibilities (e.g., allergies), medical information, or any other type of information that may be relevant to the user in connection with a given crisis. Notably, before collecting any information, in various embodiments the user can be presented with a notice of the information that may optionally be collected from the user and the purpose of collecting such information (i.e., how the information is used). The notice may include a request for consent from the user in order to track the various information that may optionally be collected (e.g., health information). The notice may include selectable objects (e.g., a button, toggle, switch, checkbox, etc.) whereby a user may select the types of information it consents to share and/or the purpose(s) for which the user consents to share such information and/or the time period(s) for which the user's consent will permit the storage/use of such information. In such embodiments, the consent a user provides may be tailored to the specific types of information and purposes for which such user feels are appropriate for the application. Additionally, the notice may periodically appear at predetermined intervals to regularly obtain consent from the user to confirm the user is aware of his privacy rights and/or to otherwise comply with privacy laws. The notice can also explain how the user's personal information is collected, used, shared, modified, anonymized, pseudonymized, minimized, secured, and/or otherwise operated upon if the user voluntarily adopts the infectious disease contact-tracing feature of the present disclosure.

In some embodiments, geolocation and/or timing data may be collected while the user is on the premises or within a zone of interest and tracking may stop (permanently or temporarily) when the user leaves the premises or zone of interest. The zone of interest may be defined, in whole or in part, by the beacons (or the locations of the beacons) existing on the premises. Additionally, the system can be capable of deriving information, drawing conclusions, forming interferences drawn from this geolocation and/or timing data, alone or together with any other information obtained from any source. For example, the system may detect a high volume of users in a particular area, and responsively provide (directly or indirectly) notifications warning users to stay away from such particular area based on the high volume of users in that area, positive reports near that area, and the like. As another non-limiting example, the system may provide users alternative routes based on positive reports sharing a common route before becoming or after becoming infected. Stated differently, the system may analyze the paths or routes of users having a positive report and infer locations in which the disease may still be present or active (e.g., as airborne particulates). The conclusions drawn and/or the inferences formed may also be based on the attributes of each disease such that a lower level of risk notification can be provided for diseases that (i) cannot survive outside the body, (ii) can only survive for a short duration, (iii) can only be spread through physical contact (or some interaction among individuals that is atypical for a given area), or (iv) other attribute.

The system may include stationary nodes and mobile nodes. Stationary nodes may be fixed in a specific location that is known to the system (e.g., beacons, gateways, or other computing devices in a known/stored location), while mobile nodes may move about from one location to another (e.g., mobile phones of users that move about). The stationary nodes in one embodiment may comprise beacons, gateways, or other nodes that, alone or together with other nodes, provide location information (e.g., a location or a micro-location) of mobile nodes that comprise smartphones. A location may include a GPS coordinate or other coordinate designating geographic location. A micro-location may include more specific details about a location, including a room, floor, or other specific details. The stationary nodes (e.g., beacons, gateways) may unidirectionally or bidirectionally communicate with mobile nodes (e.g., user devices) and securely communicate with one or more servers or cloud-computing platforms. In one embodiment, the nodes comprising stationary nodes and mobile nodes may bidirectionally communicate with Amazon Web Services (“AWS”) or similar platform over Bluetooth® Low Energy (“BLE”) or other communication protocol and/or a network. AWS provides on-demand cloud computing platforms or services that may be used to encrypt, store, and process received communications from the nodes. AWS may also transmit the processed communications received from the nodes to an administration or administer. The administration may determine whether additional steps (e.g., notify nearby mobile nodes, recommend or require quarantine of users associated with infected mobile nodes) should be taken based on the processed results.

Embodiments of the presently disclosed technology may also be used to address issues that may occur when a network (e.g., a cellular network) goes down. That is, in some embodiments the systems of the present disclosure may be built upon an established infrastructure to communicate health crisis information via various forms of transmission that can enhance redundancy, stability, and connectivity. Bidirectional communication between various types of nodes or network devices that are configured to use a multitude of communication protocols enables administrators to still notify mobile users even if one of the communication protocols goes down or is severely limited. Moreover, various nodes may be used to track the contact of users, assets, and/or zones of interest with an infectious agent—for example, bidirectional communication via mobile nodes or mobile computing devices within a proximity to each other, which may be utilized alternatively or in addition to bidirectional communication between a stationary node (e.g., beacon) and a mobile node (e.g., smartphone) when the mobile node is within a space that is proximal to the stationary node.

The presently disclosed technology is described using a number of examples in the context of the COVID-19 pandemic, and is often referred to as an “epidemic response system” in connection therewith. But it should not be interpreted to be limited to such, and could also be referred to as a “tracing system” for some embodiments. A person of ordinary skill in the art reviewing the present disclosure will understand and appreciate that the technology disclosed herein may be used to address various situations, including but not limited to: (i) the spread of any number or kind of diseases associated with infectious agents or pathogens of interest, (ii) congestion of people or vehicles carrying people in a particular area or location, (iii) congregation, congestion, vehicle traffic, walking traffic, and/or attendance evaluation (quantifying, modeling) for other purposes such as efficient route mapping, advertising, route mapping, venue/site recommendation, event attendance, payroll computations, etc.

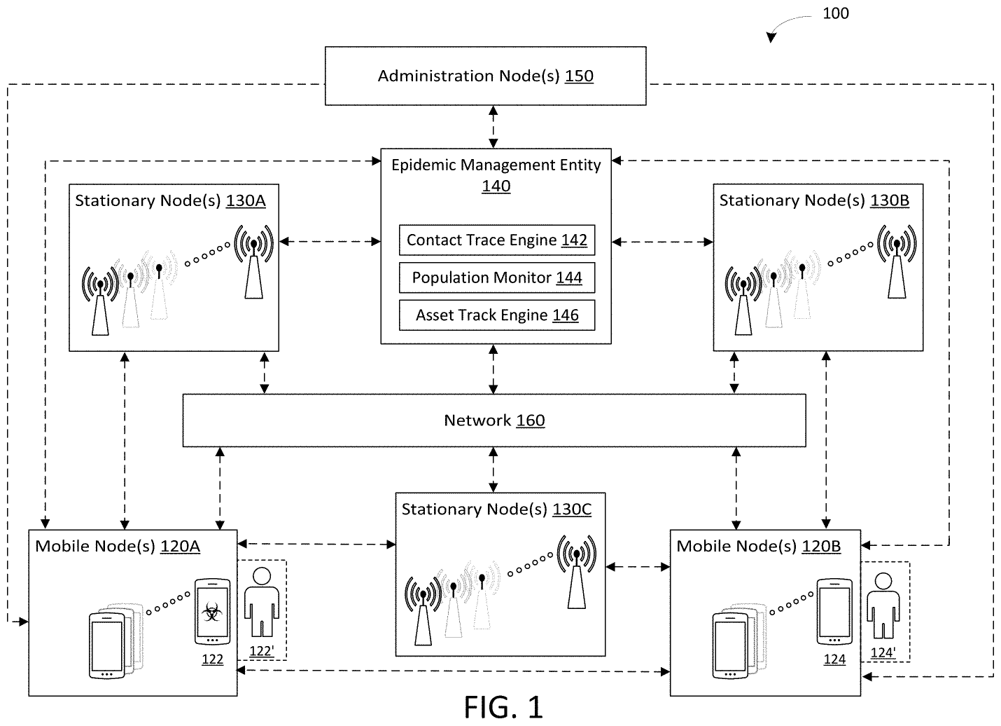

FIG. 1 is a block diagram illustrating an example epidemic response system in accordance with one or more embodiments of the disclosed technology. As shown, epidemic response system 100 (hereafter, system 100) may include one or more mobile nodes 120A-120B, one or more stationary nodes 130A-130C, and one or more administration nodes 150 in communication with an epidemic management entity 140 (hereinafter “EME 140”). Each mobile node 120A and 120B (e.g., smartphone) is associated with a user of that mobile node. As such, mobile nodes 120A-120B that are determined, by epidemic response system 100, to be exposed to an infectious agent also refers to the infected or exposed users associated with these mobile nodes. Stated differently, identified infected or exposed mobile nodes (e.g., mobile node 122) correspond to respective infected or exposed users (e.g., user 122′). Likewise, unexposed nodes (e.g., mobile node 124) correspond to respective uninfected or unexposed users (e.g., user 124′) as illustrated in FIG. 1. Due to the correspondence between the location of a user and their smartphone, in some cases mobile nodes may be understood include both the smartphones themselves and the users of those smartphones (e.g., mobile nodes 120A may be understood to include mobile node 122 and its respective user 122′, and mobile nodes 102B may be understood to include mobile node nodes 124 and its respective users 124′.

These mobile nodes 120A-120B may be in bidirectional communication with emergency management entity (“EME”) 140 which may include functionality for tracing (e.g., contact trace engine 142), population density monitoring 144, asset tracking 146, and other components. These components and their associated functionality may be embodied in non-transitory computer readable medium including instructions implemented by a processor of a mobile node (e.g., a downloaded epidemic “app”), a remote server, cloud-based services and/or a combination thereof. As a non-limiting example, processing components for contact trace engine 142, population monitor 144, and asset track engine 146 may be implemented entirely in a cloud-based platform such as Amazon Web Services (“AWS”) or similar platform. In this example, AWS may host one or more of the dedicated functions attributed to the EME 140 illustrated in FIG. 1. For example, mobile nodes 120A and 120B may be configured to transmit health, location, or other relevant data over BLE and/or a network 160 to the EME 140 hosted on AWS. The data received from the mobile nodes 120A and 120B may be used, directly or indirectly, as the input for components 142, 144, and 146. EME 140, with one or more of its components, analyzes the received data to identify events, patterns or trends concerning the data, e.g., the spread of an infectious disease. For example, the received data may be analyzed to identify events, patterns, and/or trends of users associated with mobile nodes 120A and 120B that may have been exposed to an infectious agent. As shown, the communication between one or more of: mobile nodes 120A-120B, stationary nodes 130A-130C, EME 140, and administration nodes components 150, may occur, directly or indirectly, over any one or more communications links (e.g., wired or wireless connections) including via one or more networks 160 (e.g., cellular network, Bluetooth® network, ZigBee® network, Wi-Fi® network, etc.), inclusive of the hardware and software required to establish such a communications link (e.g., communications interfaces such as cellular chipsets, Bluetooth® modules, ZigBee® modules, Wi-Fi® modules, etc.).

In some embodiments, system 100 may obtain location information of one or more mobile nodes 120A-120B within (or associated with) a zone of interest and obtain location information of one or more stationary nodes 130A-130C also within the zone of interest. In some embodiments, stationary nodes 130A-130C can be used to generate virtual boundaries that define the zone of interest. The location information may be communicated, directly or indirectly, between mobile nodes 120A and 120B as well as between stationary nodes 130A, 130B, and 130C. Furthermore, mobile nodes 120A-120B may transmit and receive information from stationary nodes 130A-130C.

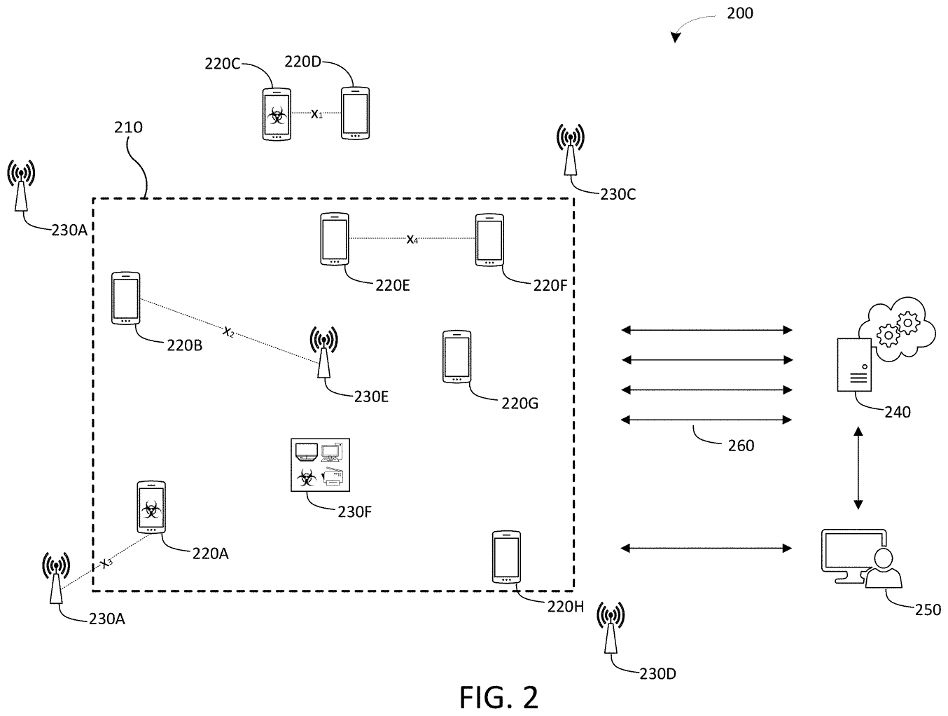

As shown in FIG. 1, mobile nodes 120A-120B may be mobile computing devices such as, for example, smartphones (illustrated in FIGS. 1 and 2), tablets, netbooks, laptop computers, or any other mobile node able to communicate over a wired or wireless network. The mobile nodes 120A-120B communicate with the EME 140 through a network 160. EME 140 may be a computing device such as a server, a database, a computer, a workstation, a computing node, a mobile telecommunications device, an electronic storage device, a computing platform, or other computing resource. EME 140 can comprise one or more servers. In various embodiments, the EME 140 may be a data center where all the one or more servers are physically co-located. Other embodiments may have the one or more servers being located in different physical locations, with each of the servers being connected in a distributed computer network. In some embodiments, at least one of the one or more servers may be a virtual server. The EME 103 may comprise a cloud server. In various embodiments, the EME 140 may comprise a combination of these different server types.

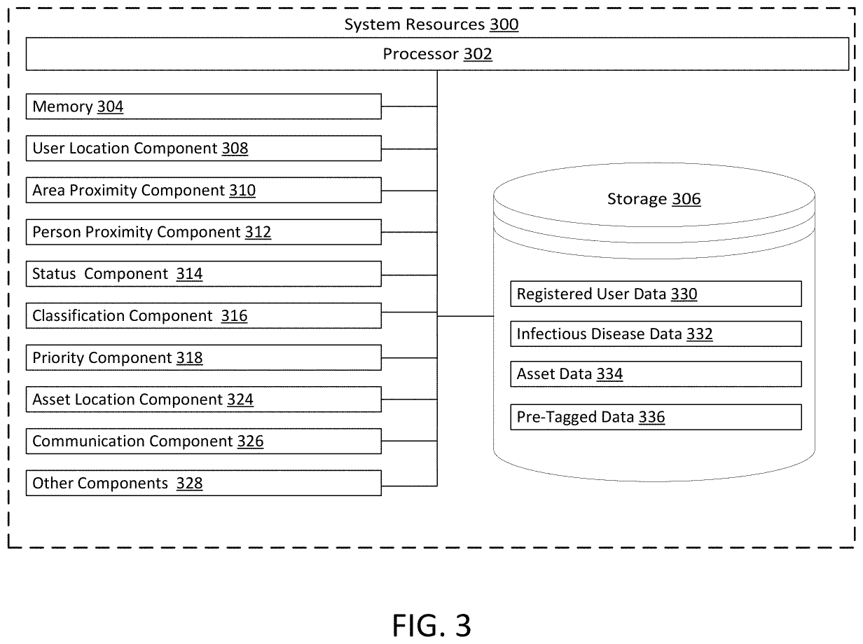

Mobile nodes 120A-120B, stationary nodes 130A-130C, and EME 140 may include one or more computing resources available to, and in some embodiments considered part of, system 100. The various features and technology disclosed herein may be effectuated by one or more of the computing resources of system 100, whether by the computing resources of a particular device (e.g., mobile node 120A), or by the computing resources of several devices performing operations in concert with one another such as may be the case in a distributed computing arrangement among the computing resources of multiple devices (duplicative, parallel, or distributed computing of both an epidemic management entity 140 and an administration node 150 function or feature). Exemplary computing resources are shown in FIG. 3 discussed herein.

Referring still to FIG. 1, mobile nodes 120A-120B may run an application such as a mobile epidemic management application (hereafter “Epidemic App”) operable to communicate or cause a communication, directly or indirectly, with one or more other elements within system 100. In some embodiments, Epidemic App is hosted on EME 140 as a virtual resource accessible to mobile nodes 120A-120B. Epidemic App may facilitate access to one or more resources by, for, from, or on behalf of the mobile nodes 120A-120B, stationary nodes 130A-130C, EME 140, administration nodes 150, and may provide, distribute, or otherwise convey data obtained via any one or more of such resources to one or more others of such resources.

In some embodiments, the Epidemic App provides a graphical user interface (“GUI”) for display on the mobile node to present information and options to the user of the mobile node, and may be further adapted to receive user input that may be conveyed throughout system 100, including for example, to EME 140, to other nodes (e.g., to an exposed mobile node 122 and exposed user 122′ within a subset of mobile nodes 120A; to an unexposed mobile node 124 and unexposed user 124′ within a subset of mobile nodes 120B, and administration nodes 150) and any other components communicatively coupled to one or more elements within system 100 for a given application. As one of ordinary skill in the art will appreciate, EME 140 may include one or more APIs providing a software interface between nodes and the resources of EME 140. In some embodiments, Epidemic App may utilize or access telephony, SMS, camera, voice, data, memory, storage, processors, biometrics and other resources of mobile nodes 120A-120B, stationary nodes 130A-130C, and/or administration nodes 150. For example, a user enrolled in the system 100 may select an icon on the GUI of the Epidemic App to open a two-way communication channel between the epidemic management entity 140 and mobile nodes 120A-120B, such as a particular mobile infected node 122 (together with user 122′) or a particular mobile uninfected node 124 (together with user 124′). Such communications may include SMS text messaging conversations, video messaging, picture messaging, audio messaging, MMS messaging, in-app messaging conversations, a voice call, a video conference, etc.

In another example, a user may select an icon or menu item on the GUI of the Epidemic App to activate a resource of an unexposed and uninfected mobile node 124 to assist the user 124′ in avoiding the exposed and infected node 122 (including user 122′) or an area (e.g., predefined set of boundaries) that the exposed node 122 is within or has recently been within. For instance, a user 124′ may activate a speaker of an unexposed node 124 to propagate an alarm or warning sound to help the user 124′ avoid the exposed node 122 (and the user 122′ associated with the exposed node 122) as they near the location identified by system 100. System 100 may be configured to provide audible or visual directions (through the aforementioned speaker or via the display of the mobile node 124) to give directions to the user to help them avoid the exposed node 122 (and the user 122′ associated with the exposed node 122) as the move about or proceed along a path toward their destination (which path and/or destination may or may not be known to the mobile node). Alternatively, an administrator node 150 associated with epidemic management entity 140 may activate a light source (e.g., an LED) of the unexposed node 124 to propagate a flashing light to help notify the user at an unexposed node 124 of their proximity to an exposed node 122 or an area (e.g., predefined set of boundaries) the exposed node 122 is within or has been within. In one embodiment, EME 140 may store and analyze past, present, and predicted locations of infected and unexposed nodes. For example, notifications directed to unexposed nodes 120 may contain information regarding locations exposed node 122 has traversed in the past 3 hours, 24 hours, the past week (e.g., a historical threshold of 1 week), or other time interval depending on the amount of information desired to be stored for a given scenario (e.g., for bacterial infections where the bacteria can survive in air particulates for up to 24 hours, the historical threshold may be set to 24 hours or longer, for instance). In another example, notifications directed to unexposed nodes 120 may contain information regarding current proximity of exposed nodes 122, previous pathways or routes upon which exposed nodes 122 traveled during a relevant time period, and predicted locations of exposed nodes 122 that should be avoided by the unexposed nodes 124.

FIG. 2 shows an example environment 200 in which one or more embodiments of the technology disclosed herein may be implemented. Specifically, FIG. 2 provides an exemplary real-world implementation of the system 100 conceptually illustrated in FIG. 1. The environment 200 may include multiple nodes or computing devices (e.g., a server/cloud services 240, user devices 220A, 220B, 220C, 220D, 220E, 220F, 220G, 220H, assets or equipment 230F, beacons 230A, 230B, 230C, 230E, 230E) and/or other components. In one embodiment, user devices 220A-220H may be mobile computing devices such as, for example, smartphones (as shown in FIGS. 1 and 2), tablets, netbooks, laptop computers, or any other mobile node able to communicate over a wired or wireless network. Any two or more components of the environment 200 may be communicatively connected to each other. Connections between the components may form a mesh network, with individual components operating as a node within the mesh network. A node within the mesh network may refer to a connection point that can receive, generate, store, and/or transmit information along one or more network routes. One or more of the mobile phones shown within environment 200 may operate as mobile nodes of the environment 200 and may be tracked to determine if they have entered, within, or left a designated area or zone of interest. The area may be defined by a geofence 210 that serves as a 2-D or 3-D virtual boundary. The geofence 210 can be created by stationary nodes (e.g., beacons, gateways, and the like) disposed at the periphery of a desired area. For example, as illustrated in FIG. 2, beacons 230A-230D may be used to define one or more borders or boundaries of geofence 210. Alternatively, a single beacon, such as beacon 230E in FIG. 2, may be used to dynamically generate the geofence 210 as a radius around the point location of beacon 230E. The area of the geofence 210 may be based on beacons already disposed on the premises of an organization, beacons placed in position for ad hoc events or meetings, and/or a combination thereof. The location coordinates of the stationary nodes may be determined with a high level of specificity (with a smaller margin of error than a mobile phone may be able to produce with its native GPS module), and the coordinates (or other location information) of such nodes may be stored in a memory location within the system 200 (e.g., a memory at servers 240 hosting an EME 140, for example). As such, the exact location of the stationary nodes may be known to the system and used as the basis for highly reliable location determinations made concerning other elements of the system such as mobile nodes (e.g., via trilateration, triangulation, signal strength derivations, signal direction information, etc.).

Once geofence 210 is established, system 220 may collect health and location information of user devices that trigger or traverse the boundaries of the established geofence 210. Simultaneously, information previously collected can be transmitted, in real-time or at predefined intervals, via a network 260 to one or more servers 240. In some embodiments, the collected information is processed offsite where an EME may be hosted (e.g., via cloud-based services such as AWS). The processed and analyzed information including identified patterns, trends, and predictions is provided to an administration or administrator node 250. The administration 250 may decide to notify (e.g., send warnings, safety recommendations, quarantine protocols, etc.) an entire organization or a few select individuals (via respective mobile nodes associated with each) based on the processed and analyzed information (e.g., health information, location information, etc.)

The user devices 220A, 220B, 220C, 220D, 220E, 220F, 220G, 220H and the asset or equipment 230F may operate as nodes within a mesh network. The topology of a mesh network may depend on the number, type, and/or communication protocol of nodes associated therewith. Additional parameters that may impact the variable topology of a mesh network can include the minimum level of redundancy or fault tolerance and/or the geographical location of the network. Additional implementations of nodes forming a mesh network may include those described in U.S. patent application Ser. No. 16/559,294, filed on Sep. 3, 2019, entitled “GEOFENCED MESH NETWORK COMMUNICATION,” the content of which is incorporated herein by reference in its entirety.

FIG. 2 shows several distance measures (e.g., X1, X2, X3, X4) that may be determined and/or collected by the system 100 to accurately identify exposed nodes 122 and evaluate when a “contact” or other traceable event has occurred between two mobile nodes. As discussed above, reference identifiers beginning with 220 depict user devices and reference identifiers beginning with 230 depict beacons. The following distances are described in reference to these categorizations and begin with (1) X1: the distance between user device 220C and user device 220D; (2) X2: the distance between user device 220B and beacon 230E; (3) X3 the distance between user device 220A and beacon 230A; (4) the distance between user device 220E and user device 220F. Distances between mobile nodes can be determined by respective locations of the mobile nodes, which may be determined in whole or in part by the mobile nodes' position/proximity relative to a stationary node, and the derived distances can be used as thresholds for a 2-layer proximity analysis (which may be critical when applied in connection with infectious distances). In embodiments, if a node is in within a close contact threshold with respect to another node, a record of the close contact is collected. The record may be associated with a user identifier (e.g., tied to a user ID of the node) stored in the EME 240. Close contact thresholds may be preset, modifiable, or dynamically adaptable based on external conditions, and can be based on the characteristics of the pathogen of interest. For example, a close contact may be defined for COVID-19 purposes as occurring when any individual who was within 6 feet of an infected person for at least 15 minutes starting from 2 days before illness onset (or prior to positive pathogen collection for asymptomatic individuals) until the time the individual is isolated. Thus, a contact threshold between mobile nodes can be 6 feet and continuous for 15 minutes. In another example, a close contact may be defined for a different disease as occurring at a first distance/time threshold if the temperature falls within a first range (e.g., a temperature range at which the pathogen can live longer) and a second distance/time threshold if the temperature falls within a second range (e.g., a temperature range at which the pathogen cannot survive for as long), and the close contact threshold may dynamically adapt based on the temperature in the relevant area (as may be measured by the mobile nodes in the zone of interest, as may be measured by the stationary nodes in the zone of interest, or as may be obtained from an external resource such as the Weather Channel® database from IBM®). Other static and/or dynamic thresholds may be used and based on distance between nodes for any purpose and based on any factor that may be relevant to the contact or tracing analysis (e.g., exposure time to the infectious agent or pathogen of concern, duration of an event, etc.).

The number of nodes within the geofence at a given time, or that have passed through the geofenced zone within a given time, may also be determined and compared to a threshold. The threshold may be set based on the number of people within or who have passed through a given space. If the number of people surpasses this threshold, an alert or notification may be provided to users and/or to EME 240 for recordation and analysis. The status of the nodes and their users (e.g., as exposed or unexposed, infected or uninfected), in various embodiments does not impact the designated number of people that are compared to the threshold number of people in a given space. The status of the nodes and their users (e.g., as exposed or unexposed, infected or uninfected), in various embodiments does impact the designated number of people that are compared to the threshold number of people in a given space.

FIG. 3 is a diagram illustrating example resources of system 100 in accordance with one or more embodiments of the disclosed technology, which may be embodied in or effectuated in whole or in part by one or more of mobile nodes 120A-120B, stationary nodes 130A-130C, epidemic management entities 130, and/or administration nodes 150 shown in FIG. 1. As shown, system resources 300 may include one or more processing engines (represented symbolically by processor 302), one or more memories (represented symbolically by memory 304), and one or more electronic storage units (represented symbolically by electronic storage 306). Memory 304 may be configured with machine readable instructions which, when executed by processor 302, cause system 100 to effectuate one or more features discussed herein, including with respect to elements identified by numerals 308-328 discussed below (which may be embodied in or effectuated by one or more of exposed nodes 122, unexposed nodes 124, mobile nodes 120A-120B, epidemic management entities 140, stationary nodes 130A-130C. System resources 300 may include one or more of a user location component 308, area proximity component 310, person proximity component 312, status component 314, classification component 316, priority component 318, population density component 320, asset location component (includes sanitization timeline) 324, communication component 326, and other components 328.

A user location component 308 in accordance with one or more embodiments of the present disclosure may be configured to obtain location data of one or more mobile nodes 120A-120B (e.g., exposed nodes 122 and unexposed nodes 124). In some embodiments such data may be obtained via location resources (e.g., location circuitry) local to such nodes, and may be provided to system 100 over network 160. User location data is indicative of the geospatial location of one or more of mobile nodes 120A-120B associated with or connected to system 100 (collectively, “units”). In some embodiments, the location of a mobile node may be determined with high degree of accuracy using triangulation and/or trilateration based on distances to one or more multiple stationary nodes. All such location determining measures (GPS+trilateration based on stationary nodes) may be considered separately or in combination.

User location component 308 may include components included in nodes (e.g., node local resources), components included in epidemic management entity 140 (e.g., communications modules, machine-readable instructions), or both. In accordance with one or more embodiments of the present disclosure, system 100 may obtain location data by actively transmitting a location data request to one or more of the units, thereby prompting the one or more units to access local location resources (e.g., GPS modules, altimeter, barometer, beacons, etc.) to obtain location information and generate and transmit a data packet containing the location data (e.g., location details) to a computing resource of system 100. For instance, EME 140 of system 100 may receive a responsive transmission from the one or more units containing the one or more units' respective location data, and system 100 may propagate and/or store such information within an electronic storage or memory of system 100 as desired for a particular application or by a particular resource coupled to or within system 100.

Location data may indicate geospatial location of a user associated with the unit, including longitude and latitude coordinates, degrees/minutes/seconds location parameters, altitude above sea level, altitude above ground level, etc. User location component 308 may be utilized to identify geospatial location of a user. User location component 308 may comprise one or more circuits, modules, or chips local to the units themselves. For example, location component 308 may include a GPS sensor, an altimeter, a pressure sensor (e.g., a barometer), and the like. In some embodiments user location component 308 may further comprise hardware and software operating on EME 140 and communicatively coupled with location sensors of one or more units.

Location data may also indicate structure specific location details of a user, including location within or upon a structure whose dimensions or other parameters are known to or derivable by system 100. For example, if a large office building implements system 100 for emergency situations such as a health crisis involving the spread of infectious disease agents, the building operator may choose to apply asset data 334 to enhance tracking of nodes. In some embodiments infected node components 140 and/or uninfected node components 150 provide proximity data as an alternative to, or as an enhancement to, the location data obtained by the location componentry local to a unit. Moreover, using stationary nodes such as beacons, proximity sensors, receivers, etc. installed throughout the building structure (on each floor, for example), that may provide structure specific location details that may be relevant to a particular application.

As a non-limiting example, beacons installed at a particular location may transmit and/or receive signals to/from units within a predefined vicinity of the beacon's location (e.g., within 20 feet of the beacon, for example), and may provide location component 308 of system 100 with information about which units are within its vicinity. Individual beacons reporting to system resources 300 may be associated with a particular location in the building (e.g., beacon may be associated with conference room D on the north side of the building on the 6th floor). Such information, including other structural or building information, may be pre-registered or otherwise provided in advance to system resources 300 as asset data 334 or electronic storage 306). This way, location information available to system 100 may be enhanced with as much detail as desired for a given application or as desired by a given operation. For instance, a beacon may be pre-registered as being associated with an elevator bay where, in one region relative to the beacon, an elevator shaft exists. Based on the timing and location information (which may include altitude information or even floor level information depending on the granularity permitted by the system resources), the system 100 may then eliminate false “contacts” be removing filtering out scenarios which did not in fact present a material risk. For example in a case where a user takes an enclosed elevator from the first floor to the tenth floor of a building without any stops in between, the user may pass within the predefined vicinity of the beacon's location (e.g., 20 feet) and within a contact tracing threshold distance (e.g., 6 feet) of another user who may be getting a drink in an elevator bay area on the fifth floor. The system 100, may evaluate the pre-registered information together with the timing and/or location information of the two mobile nodes, and determine that one of the users was located in the elevator shaft region with respect to the stationary node, and that user moved at a continuous speed without interruption from a first altitude (the first floor) to a second altitude (the tenth floor). Based on that evaluation, the system 100 may determine that the user riding the enclosed elevator up the elevator shaft did not come into the type of “contact” of concern (for a particular application) with the user getting a drink in the elevator bay. As such, system 100 may leverage both re-registered data about the vicinity together with the location information gathered in real-time, and remove traces that would otherwise yield false positives.

In still a further example, with preregistered information about the location and building within which a particular beacon is located, system 100 may be able to filter out situations where two mobile nodes are in close contact with one another, but who happen to be simply both be leaning against a dividing wall that separates two different rooms within which each user is respectively located. This way, again, traces that would otherwise yield false positives can be eliminated. Other examples will be apparent to those of skill in the art upon reviewing the present disclosure.

In still a further example, and as discussed further hereafter with respect to asset location component 324, in some embodiments stationary nodes include location beacons, proximity sensors, receivers, RFID tags, etc. installed in an indoor location where a piece of usable equipment is stowed within the building, and/or an outdoor location where a piece of usable equipment/asset is kept. Such equipment may include assets frequently touched or used by individuals. These assets may also be associated with particular rooms that experience a high level of traffic during normal business times. In some instances, such location beacons, proximity sensors, receivers, RFID tags, etc. may be affixed to the item of equipment itself (or the housing or casing associated with such equipment). Thus, tagging these rooms and/or assets will capture contact points between individuals and assets as opposed to only between individuals, and may further improve the accuracy of contact reporting or filtering in connection with building structural information (e.g., walls and other dividers, as noted above). The tags may also depend on specific disease information such as how long a disease can be contagious attached to the surfaces of assets. Notably, Epidemic App may utilize or operate one or more resources of system 100, including resources of mobile nodes 120A-120B, stationary nodes 130A-130C, EME 140, and/or administration nodes 150.

In some embodiments, multiple mobile nodes may be associated with a single user, and user location component 308 may be configured to select which of the mobile nodes is most likely to be maintained on/with the user's person. For example, if a user has a smartwatch and a smartphone both of which are operating as mobile nodes within the environments disclosed herein, the system 100 may gather location date from both mobile nodes. Although it will often be the case that the location information received from both devices by system 100 is consistent (because both are being held by the user), it may sometimes be the case that the location information received from both devices by system 100 is inconsistent (i.e., the smartwatch is in a different location than the smartphone). In circumstances where such information is inconsistent, system 100 may need to make a determination as to which mobile node more accurately reflects the user's location. System 100 may be configured to make such a determination utilizing information and data obtained from each respective mobile node.

For example, system 100 may obtain motion related data from the motion sensors embodied within each respective mobile node. Based on that information, system 100 may determine that the user's smart phone is stationary and the user's smartwatch is in motion. Based on that information, system 100 may determine that the user has set down their smart phone or otherwise misplace that, and that the location data gathered from the smartwatch should govern or otherwise inform further decisions made or conclusions drawn in connection with the contact tracing objectives of the given application.

In another example, system 100 may obtain biometrics related data from one or more biometric sensors embodied within each respective mobile node. Based on that information, system 100 may determine that the user's smart phone obtained a fingerprint reading from the user within the last minute, while the users smartwatch has not obtained a heartbeat pulse measurement from any user within the last two days. Based on that information, system 100 may determine that the user has removed their smartwatch but likely has their smart phone held close to their person, and that the location data gathered from the smart phone should govern or otherwise inform further decisions made or conclusions drawn in connection with the contract tracing objectives of the given application.

Referring still to FIG. 3, an area proximity component 310 may define a zone of interest or a bounded area which monitors when unexposed nodes 120 and/or exposed nodes 100 fall inside. The zone of interest or the bounded area may be established by existing beacons or newly tagged to particular locations (discussed further above).

Person proximity component 312 may determine the distance between mobile nodes 120A-120B, stationary nodes 130A-130C, and/or a combination thereof. In various embodiments, a threshold number, such as 6 ft, may be used, in whole or in part, to identify exposed nodes 122 and their users 122′ and/or determine the risk of exposure for mobile nodes 120A-120B. On the other hand, a population density monitoring component may be used to count of the number of nodes within a zone of interest or a bound area at a given time or during a given timeframe.

Status component 314 obtains information stored in a user profile for one or more registered users. Such information may be provided by a user when first creating a user profile upon registering with system 100 (e.g., registration and sign-up via Epidemic App). Such information may include the user's name, age, gender, height, weight, contact information (phone, email, etc.), contact information preferences, emergency contact information, family information (spouse, children, siblings, parents, etc.), employment information, skillset, health conditions or tendencies (e.g., diabetes, asthma, claustrophobia, etc.), physical disabilities (e.g., visually impaired, hearing impaired, paraplegic, etc.). Such information may also include a categorical designation. The categorical designation may be as simple as selecting either an “administrator” category or a “standard user” category. The categorical designation could also include an “infected” and “uninfected” category, and “exposed” and “unexposed” category, a “vaccinated” or “unvaccinated” category, as alternative ways to provide diagnostic information to EME 140. Any and all such information may be stored in any electronic storage available to system 100, such as in a registered user data 330 sector of electronic storage 306. Such status information may be utilized by system 100, e.g., to inform routing information, to modify notifications for higher risk or lower risk persons, to determine exposures and/or likely infection based on risk factors and/or contact threshold distances, etc.

Priority component 318 may determine a priority for one or more mobile nodes of 120A and 120B among a plurality of mobile nodes 120A-120B. Priority may be determined by applying one or more predefined priority criteria, such priority criteria based upon one or more of user location information, equipment location information, registered user detail information, and situational status information. Priority criteria may be defined in any manner. For example, priority criteria may include an expression that computes a weighted score for each mobile node under consideration, and rank the imminence of the need to assist or quarantine the mobiles based on the score. For example, a priority criteria may be given by the expression:

PRIORITY SCORE=(Factor1*w1)+(Factor2*w2)+ . . . +(FactorN*wN) [1]

For example, suppose that an implementation of system 100 considers two different factors in determining priority. Suppose, in this example, that Factor1 represents a score based on person proximity contact tracing deriving the number of instances the mobile node (i.e., node under consideration) contacts or comes within a predefined distance of other nodes or nodes in a designated time period and Factor2 represents a score based on area proximity contact tracing deriving the number of instances a node under consideration enters populated areas (e.g., as specified by geofences) in a designated time period. Such scores and/or scoring criteria may be preset or otherwise predefined and stored within system 100. Further factors may include attributes that define an infectious agent such as infectivity, pathogenicity, virulence, toxicity, invasiveness, antigenicity, mode of transmission, morbidity, mortality, etc. These attributes can be used in combination with the proximity scores of Factor1 and Factor2 (e.g., data from person proximity component 312 and area proximity component 310) to determine the impact a particular infectious disease can have on public health and thus increase the accuracy of the calculated priority score.

For example, suppose the following scores are associated with the following number of instances within which a node under consideration must be isolated or quarantined (e.g., for Factor1):

TABLE 1.0

| Instances of Contact | Score |
|---|---|
| 50 ≤ # ≤ 40 | 5 |
| 40 < # ≤ 30 | 4 |
| 30 < # ≤ 20 | 3 |
| 20 < # ≤ 10 | 2 |
| 10 < # ≤ 1 | 1 |
| # < 0 | 0 |

System 130 may apply the scoring expression given in Eqn. 1 above, for example, and determine the priority scores accordingly.

Asset location component 324 in accordance with one or more embodiments of the present disclosure obtains equipment location data of one or more pieces of equipment in the vicinity of the emergency (the vicinity defined as desired for a given implementation). In some embodiments, such equipment location data may be obtained via location resources (e.g., location circuitry) local to or coupled with pieces of such equipment, and may be provided to system 100 over network 160 (which in some instances may be include mesh network options). Equipment location data is indicative of the geospatial location of one or more pieces of equipment in the vicinity of the emergency.

Asset location component 324 may include components coupled with or near equipment (i.e., equipment local resources), components included in emergency management entity (e.g., communications modules, machine-readable instructions, etc.), or both. In accordance with one or more embodiments of the present disclosure, system 100 may obtain location data by actively transmitting a location data request to one or more location resources coupled with or near pieces of equipment, thereby prompting the one or more location resources (e.g., GPS modules, altimeter, barometer, beacons, RFID tags, etc.) to obtain location information and cause a data packet containing the location data (e.g., location details) to be generated and/or transmitted to a computing resource of system 100. For instance, EME 140 of system 100 may receive a responsive transmission from the one or more pieces of equipment (i.e., from a communication interface coupled with a piece of equipment) containing the one or more pieces of equipment's respective equipment location data, and system 100 may propagate and/or store such information within an electronic storage or memory of system 100 as desired for a particular application or by a particular resource coupled to or within system 100.

Asset location data may indicate geospatial location of a piece of equipment in the vicinity of the emergency, including longitude and latitude coordinates, degrees/minutes/seconds location parameters, altitude above sea level, altitude above ground level, etc. Asset location component 324 may be utilized to identify geospatial location of a piece of equipment. Asset location component 324 may comprise one or more circuits, modules, or chips local to the units themselves. For example, Asset location component 324 may include a GPS sensor, an altimeter, a pressure sensor (e.g., a barometer), beacon (e.g., Bluetooth® beacon), RFID tag, and the like. In some embodiments, Asset location component 324 may further comprise hardware and software operating on EME 140 and communicatively coupled with location sensors of one or more units.

Classification component 316 may provide the categorization of a plurality of nodes based on each nodes level of exposure to infectious agents. For example, nodes may be tagged as exposed nodes, unexposed nodes, exposed nodes, or recovered nodes based on at least the user location component 308 and registered user detail component 314. User location component 308 may determine the location of user associated with a particular node and registered user detail component 314 that can include an imported self-diagnosis, present (if any) symptoms, and other relevant health attributes of user (e.g., user has antibodies for the infectious disease). The classification component may be capable of more granular classifications if data from the area proximity 310 and person proximity 312 components is also inputted into the classification component.

The classification can also triage (sort and classify) nodes to determine priority and type of notifications relayed to these nodes and other nodes in proximity to these nodes. During infectious disease outbreaks, triage is particularly important to separate nodes likely to be infected with the pathogen of concern. In one embodiment, mobile nodes may be classified as infected when (1) diagnosis was confirmed by a positive lab test and/or (2) clinical diagnosis was made by a healthcare provider, (3) patient or caregiver suspects COVID-19 (or other disease) based on symptoms consistent with COVID-19 (or other disease) and living in an area of community spread, and/or any other classification scheme desired for a given disease.

Symptoms consistent with COVID-19 include cough, fever, shortness of breath. Increased age is also a strong risk factor for severe COVID-19 illness and complications. Based on the results of the triage, exposed nodes may further be classified in accordance with the severity of the symptoms. In response to the severity classification by the classification component, processor 300 generates corresponding reports or notifications for the user or organization associated with the infected node. That is, the severity of the symptoms can customize the content and timing of the associated reports and notifications. Example notifications to an individual (listed decreasing in severity) may comprise Call EMS 911 Now, Go to ED Now, Call PCP Now, Call PCP within 24 hours, Call PCP when Office is Open, and Home Care. Analogous notifications can also be provided to an organization depending on the situational status and relationship of the user to the organization. As to the modified timing of the reports or notifications, the higher the severity classification, the sooner the reports or notifications are relayed to the relevant nodes. Please not that while the presently disclosed technology is described in the context of the COVID-19 pandemic in certain examples. It should not be interpreted to be limited to such. A person of ordinary skill in the art would understand how to use the techniques disclosed for effectively addressing various diseases associated with infectious agents.

An example of other components 328 may include sanitation tracking component that is discrete from the asset location component 324 further described above. The sanitation tracking component may establish timestamps for when equipment or assets of an organization have been sterilized and based on these timestamps indicate when additional sterilization is necessary. For example, a predetermined period of time following sterilization such as a week, month, or year. The predetermined period of time may be varied in accordance with the type of asset and/or status of the outstanding epidemic. The timestamps of sanitization tracking component 328 may also provide a digital record of when an asset has been exposed or may have been exposed to infectious agents (e.g., exposed nodes 122) regardless of when the asset was last cleaned to update a sterilization status of an asset and/or schedule additional sterilization.

A further example of other components 328 can comprise a reporting or component that provides a report of nodes, areas, locations, assets, and/or users that are infected. The report may be used to notify a plurality of users or a subset of users of how many people are affected in a particular area or location. The report can also indicate geographical locations including routes to these geographical locations that should be avoided.

While primarily described as discrete components, one or more of the components described above can be used, alone or in combination, by organizations seeking to enable a more proactive and comprehensive response to infectious diseases during a health crisis.

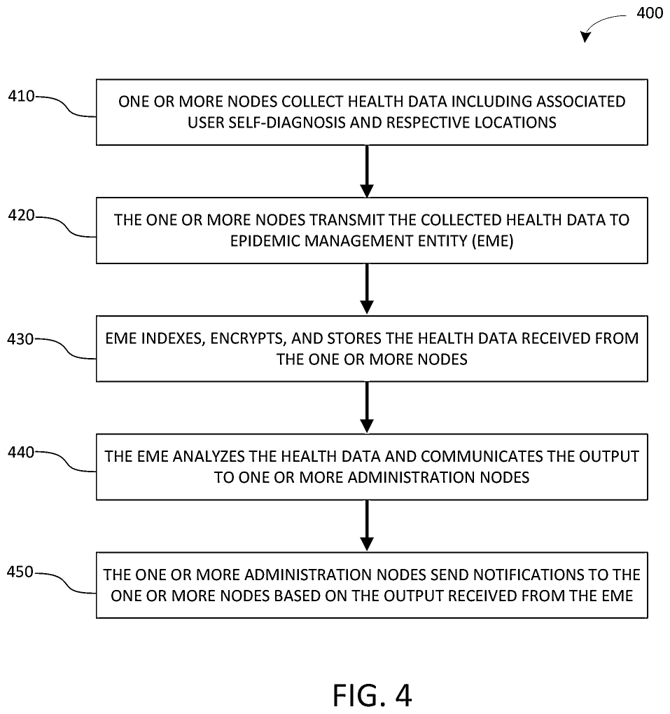

FIG. 4 is a flow diagram illustrating an example method in accordance with one or more embodiments of the present disclosure. Referring to FIG. 4, method 400 describes steps for infectious disease contact tracing. Following receiving consent by the user associated with a particular node for tracing, infectious disease contact tracing may begin. At 410, health data associated with an infectious disease is collected by a node. A node generally describes a device that is configured to receive and transmit data over a wired or wireless connection. Some nodes may be held or co-located with respective individuals while other nodes may include traditional networking devices. Alternatively, nodes may be differentiated by their mobility. That is, mobile nodes and stationary nodes. Specific examples of mobile nodes can include smartphones, tablets, a netbooks, a laptop computers, and mobile computing devices. Whereas examples of stationary nodes may include access points (“AP”), beacons, servers, routers, modems, and other analogous network devices. Though stationary nodes may be movable in that they may be installed in one location and then later moved and reinstalled in another location, these types of nodes are considered stationary in that they are generally fixed in a known location during desired operation of the technology of the present disclosure.

As to collection or storage of health data related to the infectious disease, this data broadly encompasses the general indicators of health (e.g., self-diagnosis, symptoms, health records, etc.) in addition to data indicating the geographical position or movement of a node (e.g., node location, node movement, node routes). Heath data defining the geographical position or movement of a node can be used to trace the contacts of the node and spread of an infectious disease. The position and movement data is described as health data because this data is used to assess the spread of pathogens and only for this purpose. Nevertheless, periodic consent notifications may be provided by the system 100 to confirm approval and acknowledgment of the registered users.

Next, at operation 420, the collected health data from the node is transmitted to an epidemic management entity 140. In one embodiment, cloud computing platforms, such as Amazon Web Services, relaying on datacenters for high performance computing may be utilized but other entities may also be implemented in alternative embodiments of the presently disclosed technology. The specific characteristics of the entity will vary depending on the size, connectivity, and processing power needed to analyze nodes associated with an organization. In operation 430, the database analyzes the health data received from the node to derive patterns hidden within health data transmitted from a network of nodes. Analyzation may further include running reports of employee locations during a specified time range or period. For example, during work hours or times of the day with increased levels of congestions such as rush hour in the morning and evening. The specified time range may also include days of the week or during a particular event in which it is likely employees may be exposed to an infectious disease. Relevant instances include work-sponsored events and/or gathering of a large group for meetings or personally reaching out to clients. At operation 440, the database transmits the analyzed health data including patterns, educated interferences, and predictions to an administration. The raw health data initially received at the database may be encrypted and segregated from administrators to address privacy concerns and increase the likelihood that users consent to the collection of their data that may be used to hinder the spread of a pathogen of concern. Lastly, in operation 450, an administrator communicates analyzed health data to a mobile node or nodes. The administrator refers to individuals that decide the protocols to be implemented during a health crisis. For example, a human resources department, CEO, and/or office administrator may be granted access to the health data of individuals they are responsible for and decide next steps regarding mobilized notifications. The notifications may comprise tailored recommendations corresponding to the level of risk (i.e., priority), type of employee, and determined location of the employee. Step 410 may include asset tracking to confirm sanitization of infected zones. That is, a designated building or zone exposed to an infected node and logging subsequent remedial measures. A historical record for assets or pieces of equipment may be stored in the memory of various components of the presently disclosed technology enabling reminders and up-to-date information on the infection status of a particular location or piece of equipment.

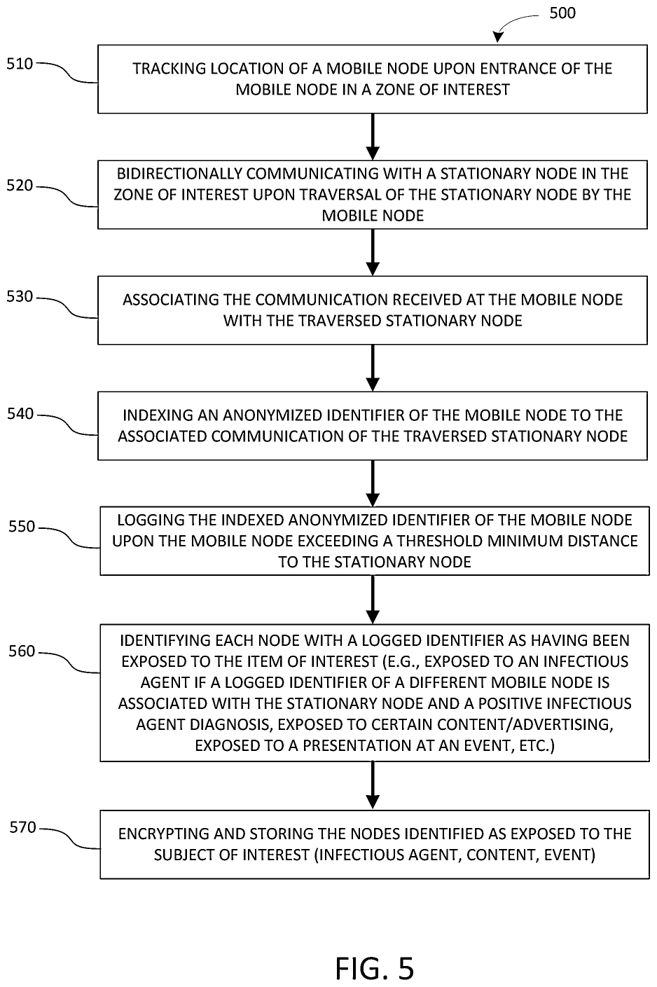

FIG. 5 is a flow diagram illustrating an example method in accordance with one or more embodiments of the present disclosure. Referring to FIG. 5, method 500 comprises operations for performing contact tracing (e.g., in connection with an event, a business or government interest, or an infectious disease such as COVID-19). In step 510, the system of the presently disclosed technology may start tracking the location a node when the node enters a zone of interest. The zone of interest may be an area that is defined by a geofence. As shown in FIG. 2, a geofence establishes a virtual barrier around an area. The geofence is useful for triggering certain events after a tracked device enters and/or leaves a particular area. For example, tracking of the node entering a location such as a work building may be triggered by the user associated with the mobile node walking into his workplace.

In step 520, movement of the node passing another node is tracked. The other node may be a stationary node (e.g., beacon) or other device capable of receiving and transmitting data. In other embodiments, the stationary node may be another mobile node and relative movement between the two may be detected. In step 530 the system data transferred between the stationary and mobile node with a particular mobile node. Such that, the system will be able to determine that the mobile node passes a particular stationary node. In this way, the location of the mobile node may be determined because the location of the stationary node is already known. Next, at 540, the system indexes a stored identifier for the mobile node with the stationary node and communication from the stationary node. In step 550, the derived position of the mobile node is compared to the position of the stationary node. The distance between the two nodes is then compared to a distance threshold. If there is close contact between the stationary node and the mobile node, the comparison is logged. Next, at 560, the logged close contact is checked against other instances of closed contact with the stationary node (in embodiments directed toward infectious agent transmission, if there are other instances of close contact, the mobile node previously indicated as uninfected, is now classified as exposed to an infectious agent). Thereafter, the classification is stored checked against other logged comparisons that has previously been stored in the resources of the system.

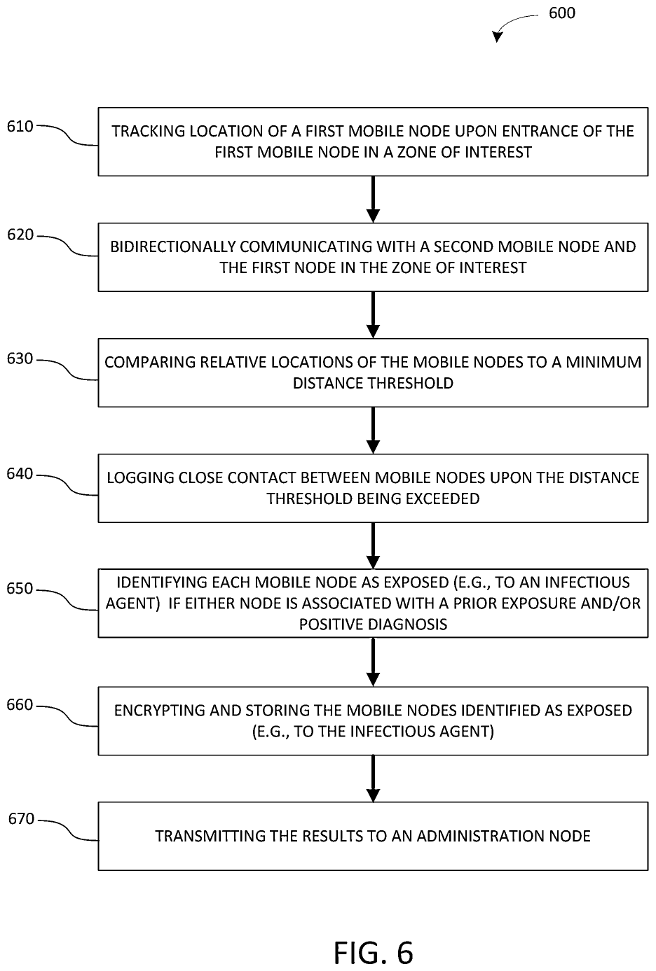

FIG. 6 is a flow diagram illustrating an example method in accordance with one or more embodiments of the present disclosure. Referring to FIG. 6, method 600 comprises operations for performing contact tracing of an infectious disease (e.g., COVID-19). However, method 500 provides steps for an area proximity between a stationary node and a mobile node, while method 600 provides steps for a person proximity between a mobile node and a mobile node. Method 600 requires less steps than method 600 because it is not necessary to determine which stationary node the mobile node has traversed. Mobile nodes are associated with respective users that have already consented to having their locations tracked. Moreover, their respective locations are already linked to particular anonymized user IDs stored in the system (which may be, modified, pseudonymized, minimized, secured, and/or otherwise operated upon). Referring to FIG. 6, at 610, as a mobile node enters an area it is automatically tracked by the system. In step 620, mobile nodes within the area or within the same zone of interest communicate with each other. The communication may be bidirectional and encompass various mediums. For example, each mobile node may utilize BLE and/or Wifi to communicate with each other. After communicating with each other and thus indicating to the system that they are within a proximity to each other, the distance between the mobile nodes is compared to a distance threshold. In step 640, the mobile nodes have exceeded the threshold minimum distance and this result is stored by the system. Thereafter, at 650, the system determines whether either of the mobile nodes is associated with a positive diagnosis for an infectious disease. If either mobile node is associated with an infectious disease, at 660, the mobile node that was previously uninfected, is identified as exposed to the infectious disease. In 670, the updated status or diagnosis information of the mobile node with a new status is then stored and transferred to an administration for further handling. The administration may decide to notify the newly identified exposed mobile node immediately for self-quarantine purposes.

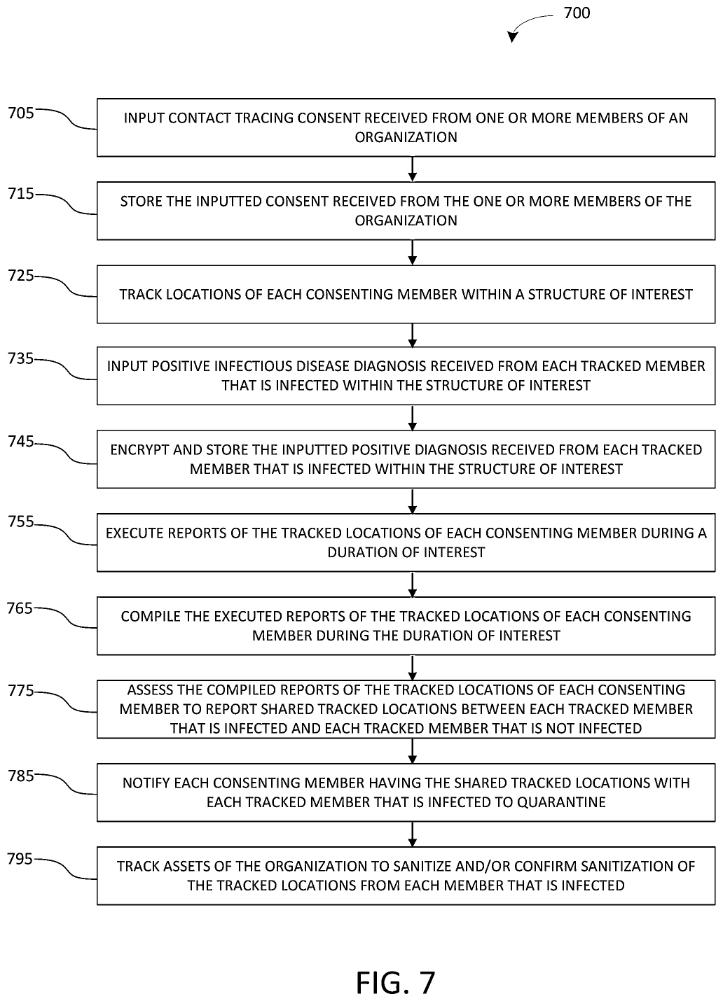

FIG. 7 is a flow diagram illustrating an example system in accordance with one or more embodiments of the present disclosure. Referring to FIG. 7, at 705, the system 700 can input contact received from one or more members of an organization. Next, at 715, the system 700 may store the inputted consent received from the one or more members of the organization. In this way, the system 700 can track locations of each consenting member within a structure or zone of interest. The system 700 may further, in step 735, input positive infectious disease information or diagnoses received from each tracked member that is infected within the structure of interest. Following collection of various forms of input, the system 700, in 745, is configured to encrypt and store the inputted positive diagnoses received from each tracked member that is infected within the structure of interest. Then, at 755, reports may be executed describing the tracked locations of each consenting member during a duration of interest. In response to execution of the reports, at 765, the executed reports may be compiled for assessment at 775. The assessed reports showing shared tracked locations of members of an organization may be used as the basis to notify relevant members of the organization. Specifically, at 785, the system 700 may notify each consenting member having the shared tracked locations with each tracked member that is infected to go into quarantine. Lastly, at 795, the system 700 may further be configured to track assets of the organization to sanitize and/or confirm sanitation of the tracked locations from each member that is infected.

Referring to FIGS. 1-7 collectively, various status identifiers are often used with respect to a mobile node, including an “infected” or “uninfected” node, an “exposed” or “unexposed” node, or a “vaccinated” or “unvaccinated” node. Each of these are merely examples of different status identifiers for the respective users of such nodes. And while there are instances in this disclosure where just one or two of these status identifiers may be referred to in a particular example, the examples should not be considered to be limited to the one or two status identifiers referred to. For instance, where an example herein refers to an “infected” or “uninfected” mobile node, the disclosure should be read to also include the same example in connection with an “exposed” or “unexposed” mobile node, as well as the same example in connection with a “vaccinated” or “unvaccinated” node as the case may be, or any other status indicator (“recovered”, “in treatment”, taking “[x] drug” etc.). And indeed, each example provided herein should be understood to extend to scenarios where all such identifiers may be used alone or in combination. For instance, a particular mobile node may be considered “exposed” but “uninfected.” As another example, a particular mobile node may be considered “exposed” and “infected” but “recovered.” As another example, a particular mobile node may be considered “exposed” but “previously vaccinated” and thus deemed “uninfected.”

Referring still to FIGS. 1-7 collectively, any and all of the information obtained, analyzed, derived, determined, created, conveyed or transmitted, within the systems or in accordance with the methods disclosed herein (e.g., user IDs, user device IDs, user location, location information, building information, user personal information, user names, etc.) may be maintained in raw, original or otherwise complete form, or, in some embodiments, one or more of such information may be anonymized, pseudonymized, or data minimized before or after being obtained, analyzed, derived, determined, created, or conveyed, or transmitted. In some embodiments, one or more of such information may further be encrypted.

Referring still to FIGS. 1-7 collectively, although these illustrate example embodiments with components, elements and circuits partitioned in the depicted manner, it will be appreciated by one of ordinary skill in the art that various components and circuits of system 100 (including any one or more elements and subsystems related thereto, individually or collectively) and described herein may be implemented utilizing any form of hardware, software, and/or a combination thereof. For example, one or more processors, controllers, ASICs, PLAs, PALs, CPLDs, FPGAs, logical components, software routines or other mechanisms, including associated memory, might be used to implement one or more components or circuits in embodiments of system 100 (including any one or more elements and subsystems related thereto) described in the present disclosure. In embodiments, the various components and circuits described herein might be implemented as discrete components or the functions and features described can be shared in part or in total among two or more components. In other words, as would be apparent to one of ordinary skill in the art after reading this description, the various features and functionality described herein may be implemented in any given application and can be implemented in one or more separate or shared components in various combinations and permutations. Even though various features or elements of functionality may be individually described or claimed as separate components, in various embodiments these features and functionality can be shared among one or more common software and hardware elements, and such description shall not require or imply that separate hardware or software components are used to implement such features or functionality.

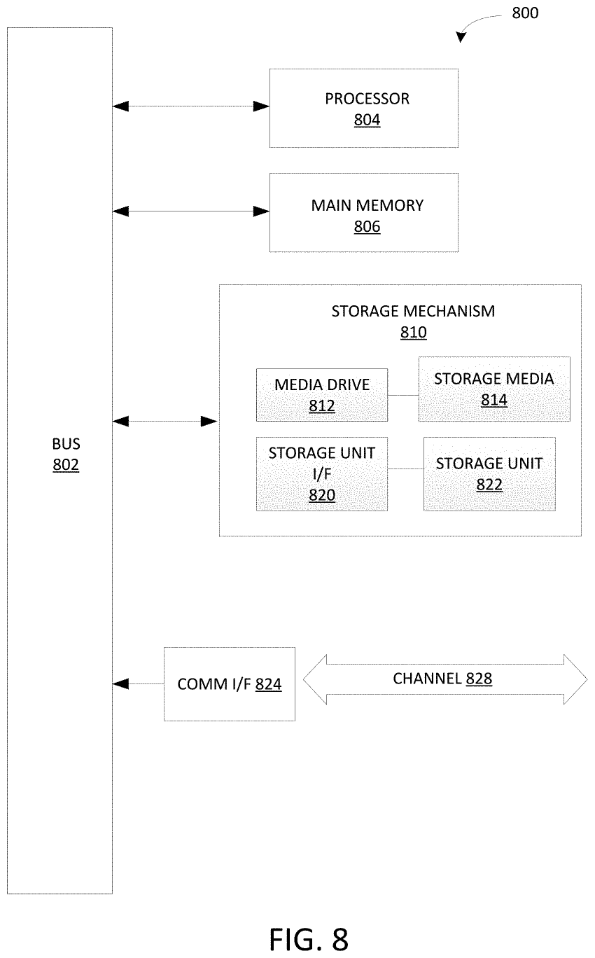

As used herein, the term circuit/logic might describe a given unit of functionality that can be performed in accordance with one or more embodiments of the technology disclosed herein. As used herein, a circuit/logic might be implemented utilizing any form of hardware, software, firmware, and/or a combination thereof. For example, one or more processors, controllers, ASICs, PLAs, PALs, CPLDs, FPGAs, logical components, software routines or other mechanisms might be implemented to make up a circuit/logic. In implementation, the various circuits/logics described herein might be implemented as discrete circuits/logics or the functions and features described can be shared in part or in total among one or more circuits/logics. In other words, as would be apparent to one of ordinary skill in the art after reading this description, the various features and functionality described herein may be implemented in any given application and can be implemented in one or more separate or shared circuits/logics in various combinations and permutations. Even though various features or elements of functionality may be individually described or claimed as separate circuits/logics, one of ordinary skill in the art will understand that these features and functionality can be shared among one or more common software and hardware elements, and such description shall not require or imply that separate hardware or software components are used to implement such features or functionality.

Where components or circuits/logics of the technology are implemented in whole or in part using software, in one embodiment, these software elements can be implemented to operate with a computing or processing circuit/logic capable of carrying out the functionality described with respect thereto. One such example computing circuit/logic is shown in FIG. 8. Various embodiments are described in terms of this example-computing circuit 800. After reading this description, it will become apparent to a person skilled in the relevant art how to implement the technology using other computing circuits/logics or architectures.

Referring now to FIG. 8, computing circuit 800 may represent, for example, computing or processing capabilities found within desktop, laptop and notebook computers; hand-held/wearable computing devices (PDA's, smart phones, smart glasses, cell phones, palmtops, etc.); mainframes, supercomputers, workstations or servers; or any other type of special-purpose or general-purpose computing devices as may be desirable or appropriate for a given application or environment. Computing circuit 800 might also represent computing capabilities embedded within or otherwise available to a given device. For example, a computing circuit might be found in other electronic devices such as, for example, digital cameras, navigation systems, cellular telephones, portable computing devices, modems, routers, WAPs, nodes and other electronic devices that might include some form of processing capability.

Computing circuit 800 might include, for example, one or more processors, controllers, control circuits, or other processing devices, such as a processor 804. Processor 804 might be implemented using a general-purpose or special-purpose processing engine such as, for example, a microprocessor, controller, or other control logic. In the illustrated example, processor 804 is connected to a bus 802, although any communication medium can be used to facilitate interaction with other components of computing circuit 800 or to communicate externally.

Computing circuit 800 might also include one or more memory components, simply referred to herein as main memory 808. For example, preferably random access memory (RAM) or other dynamic memory, might be used for storing information and instructions to be executed by processor 804. Main memory 808 might also be used for storing temporary variables or other intermediate information during execution of instructions to be executed by processor 804. Computing circuit 800 might likewise include a read only memory (“ROM”) or other static storage device coupled to bus 802 for storing static information and instructions for processor 804.

The computing circuit 800 might also include one or more various forms of information storage mechanism 810, which might include, for example, a media drive 812 and a storage unit interface 820. The media drive 812 might include a drive or other mechanism to support fixed or removable storage media 814. For example, a hard disk drive, a floppy disk drive, a magnetic tape drive, an optical disk drive, a CD or DVD drive (R or RW), or other removable or fixed media drive might be provided. Accordingly, storage media 814 might include, for example, a hard disk, a floppy disk, magnetic tape, cartridge, optical disk, a CD or DVD, or other fixed or removable medium that is read by, written to or accessed by media drive 812. As these examples illustrate, the storage media 814 can include a computer usable storage medium having stored therein computer software or data. For example, one or more memory components may include non-transitory computer readable medium including instructions that, when executed by the processor 804, cause the computing circuit 800 to perform one or more functionalities described herein.

In alternative embodiments, information storage mechanism 810 might include other similar instrumentalities for allowing computer programs or other instructions or data to be loaded into computing circuit 800. Such instrumentalities might include, for example, a fixed or removable storage unit 822 and an interface 820. Examples of such storage units 822 and interfaces 820 can include a program cartridge and cartridge interface, a removable memory (for example, a flash memory or other removable memory component) and memory slot, a PCMCIA slot and card, and other fixed or removable storage units 822 and interfaces 820 that allow software and data to be transferred from the storage unit 822 to computing circuit 800.

Computing circuit 800 might also include a communications interface 824. Communications interface 824 might be used to allow software and data to be transferred between computing circuit 800 and external devices. Examples of communications interface 824 might include a modem or softmodem, a network interface (such as an Ethernet, network interface card, WiMedia, IEEE 802.XX or other interface), a communications port (such as for example, a USB port, IR port, RS232 port Bluetooth® interface, or other port), or other communications interface. Software and data transferred via communications interface 824 might typically be carried on signals, which can be electronic, electromagnetic (which includes optical) or other signals capable of being exchanged by a given communications interface 824. These signals might be provided to communications interface 824 via a channel 828. This channel 828 might carry signals and might be implemented using a wired or wireless communication medium. Some examples of a channel might include a phone line, a cellular link, an RF link, an optical link, a network interface, a local or wide area network, and other wired or wireless communications channels.

In this document, the terms “computer program medium” and “computer usable medium” are used to generally refer to media such as, for example, memory 808, storage unit 820, media 814, and channel 828. These and other various forms of computer program media or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions might enable the computing circuit 800 to perform features or functions of the disclosed technology as discussed herein.

While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that can be included in the disclosed technology. The disclosed technology is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the technology disclosed herein. Also, a multitude of different constituent circuit names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “circuit” does not imply that the components or functionality described or claimed as part of the circuit are all configured in a common package. Indeed, any or all of the various components of a circuit, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A system comprising:
a non-transitory computer readable medium storing machine readable instructions which, when executed by a processor, cause the system to:
determine a timestamped location of a first mobile node based at least in part on a known location of at least one stationary node;
determine a timestamped location of a second mobile node based at least in part on a known location of at least one stationary node;
obtain identity data of a user associated with the second mobile node, the identity data comprising at least one of: a self-screening report of the user associated with the second mobile node, an exposure status of the user associated with the second mobile node, and an organizational status of the user associated with the second mobile node;
determine if a user associated with the first mobile node and the user associated with the second mobile node have come within a contact threshold distance of one another during a contact threshold timeframe based on the timestamped location of the first mobile node and the timestamped location of the second mobile node;
determine whether the user associated with the first mobile node has been exposed to an infectious disease by the user associated with the second mobile node, the determination based on one or more of the self-screening report of the user associated with the second mobile node and the exposure status of the user associated with the second mobile node; and
provide, upon determining that the user associated with the first mobile node has been exposed to an infectious disease by the user associated with the second node, a notification for display on a touchscreen display of the first node, the notification indicating a risk of exposure to an infectious agent and a corresponding safety recommendation.

2. The system of claim 1, wherein the timestamped locations of the mobile nodes are obtained from one or more of: components internal to a housing of each mobile node; components external to a housing of each mobile node; selection input from a user of each mobile node; textual input from a user of each mobile node; voice input from a user of each mobile node; and video input from a user of each mobile node.

3. The system of claim 1, wherein the known location of at least one stationary node comprises a workplace and the first mobile node and the second mobile node comprises a mobile computing device that corresponds to an employee of the workplace.

4. The system of claim 1, wherein the first mobile node and the second mobile node comprise a plurality of mobile nodes, each mobile node corresponding to a mobile computing device and a user of the mobile computing device.

5. The system of claim 1, wherein the first mobile node and the second mobile node comprise at least one of a smartphone, a tablet, a netbook, a laptop computer, and a mobile computing device, wherein the first mobile node and the second mobile node are configured to communicate over a wired or wireless network and Bluetooth.

6. A method comprising:
determining a timestamped location of a second mobile node based at least in part on a known location of at least one stationary node;
obtaining identity data of a user associated with the second mobile node, the identity data comprising at least one of: a self-screening report of the user associated with the second mobile node, an exposure status of the user associated with the second mobile node, and an organizational status of the user associated with the second mobile node;
determining if a user associated with the first mobile node and the user associated with the second mobile node have come within a contact threshold distance of one another during a contact threshold timeframe based on the timestamped location of the first mobile node and the timestamped location of the second mobile node;
determining whether the user associated with the first mobile node has been exposed to an infectious disease by the user associated with the second mobile node, the determination based on one or more of the self-screening report of the user associated with the second mobile node and the exposure status of the user associated with the second mobile node; and
providing, upon determining that the user associated with the first mobile node has been exposed to an infectious disease by the user associated with the second node, a notification for display on a touchscreen display of the first node, the notification indicating a risk of exposure to an infectious agent and a corresponding safety recommendation.

7. The method of claim 6, further comprising:
generating a set of virtual boundaries based in part on the known location of the at least one stationary node, the at least one stationary node comprising a plurality of proximity beacons; and
in response to the user associated with the first mobile node traversing the generated set of virtual boundaries, determining whether the user associated with the first mobile node has been exposed to an infectious disease; and
in response to the user associated with the second mobile node traversing the generated set of virtual boundaries, determining whether the user associated with the second mobile node has been exposed to an infectious disease;
wherein the generated set of virtual boundaries form a bounded area of interest.

8. The method of claim 7, wherein one or more proximity beacons of the plurality of proximity beacons correspond to assets with the bounded area of interest.

9. The method of claim 6, further comprising:
collecting personal identifying information (PII) data of a plurality of mobile nodes;
anonymizing the PII data;
analyzing the anonymized information of the plurality of nodes;
transmitting a first notification to a first subset of nodes exposed to an infectious agent,
transmitting a second notification to a second subset of nodes likely exposed to an infectious agent;
transmitting a third notification to a third subset of nodes possibly exposed to an infectious agent;
transmitting a fourth notification to a fourth subset of nodes unlikely to be exposed to an infectious agent; and
monitoring the plurality of nodes and updating the subsets of the plurality of nodes associated with respective notifications, the updating based on one or more of self-diagnoses, health records, immunity status, recover status, antibodies, time of day, location, historical movement, and current movement, wherein the updated notifications are configured to be automatic and in real-time.

10. The method of claim 6, further comprising:
tracing contacts of a first plurality of nodes;
determining the traced contacts of the first plurality of nodes associated with a second plurality of nodes,
identifying the determined contacts of the second plurality of nodes exposed based on the distance measure and the relational measure; and
tracing contacts of a third plurality of nodes.

11. The method of claim 6, wherein the first mobile node and the second mobile node comprise at least one of a smartphone, a tablet, a netbook, a laptop computer, and a mobile computing device, wherein the first mobile node and the second mobile node are configured to communicate over a wired or wireless network and Bluetooth.

12. The method of claim 6, wherein the known location of at least one stationary node comprises a workplace and the first mobile node and the second mobile node comprises a mobile computing device that corresponds to an employee of the workplace.

13. The method of claim 6, wherein the first mobile node and the second mobile node comprise a plurality of mobile nodes, each mobile node corresponding to a mobile computing device and a user of the mobile computing device.

14. A computer-implemented method for responding to an infectious disease comprising:
inputting consent received from one or more members of an organization;
storing the inputted consent received from the one or more members of the organization;
tracking locations of each consenting member within a structure of interest;
inputting positive infectious disease diagnosis received from each tracked member that is infected within the structure of interest;
storing the inputted positive diagnosis received from each tracked member that is infected within the structure of interest;
executing reports of the tracked locations of each consenting member during a duration of interest;
compiling the executed reports of the tracked locations of each consenting member during the duration of interest;
assessing the compiled reports of the tracked locations of each consenting member to report shared tracked locations between each tracked member that is infected and each tracked member that is not infected; and
notifying each consenting member having the shared tracked locations with each tracked member that is infected to quarantine.

15. The computer-implemented method of claim 14, wherein the structure of interest comprises a worksite associated with the members of the organization.

16. The computer-implemented method of claim 14, further comprising:
tracking assets of the organization, wherein tracking comprising a record of sanitization of the tracked assets;
comparing the tracked assets to the tracked locations from each tracked member that is infected within the structure of interest;
tagging the shared locations of the tracked assets and the tracked locations from each tracked member that is infected for sanitization; and
notifying the organization of the tracked assets in the tagged shared locations that require sanitization.

17. The computer-implemented method of claim 14, wherein assessing further comprises comparing historical tracked locations of each member that is infected to a plurality of stationary nodes based on a first proximity threshold to the plurality of stationary nodes.

18. The computer-implemented method of claim 17, wherein each member of the organization corresponds to a mobile node of a plurality of mobile nodes; and
each mobile node comprises at least one of a smartphone, a tablet, a netbook, a laptop computer, and a mobile computing device, wherein each node is configured to communicate over a wired or wireless network.

19. The computer-implemented method of claim 18, further comprising:
indexing each mobile node of the plurality of mobile nodes to each stationary node of the plurality of stationary nodes if a mobile node transverses a stationary node,
identifying the indexed mobile nodes within the first proximity threshold, wherein the proximity threshold comprises a predetermined distance between the mobile node and the stationary node;
identifying each mobile node of the plurality of mobile nodes within a second proximity threshold, wherein the second proximity threshold comprises a predetermined distance between each mobile node; and
classifying each mobile node of the plurality of mobile nodes based on each member that is infected corresponding a mobile node that is identified as within the first proximity threshold or the second proximity threshold.
